(19) United States
(12) Patent Application Publication
(10) Pub. No.: US 2002/0065608 A1
(43) Pub. Date: May 30, 2002

(54) Analyzing molecular diversity by total pharmacophore diversity

(76) Inventor: Gergely M. Makara, Quincy, MA (US)

Correspondence Address: HALE AND DORR, LLP, 60 STATE STREET, BOSTON, MA, 02109

(21) Appl. No.: 09/996,114

(22) Filed: Nov. 28, 2001

Related U.S. Application Data
(60) Provisional application No. 60/253,835, filed on Nov. 29, 2000.

(51) Int. Cl. G06F 19/00; G01N 33/48; G01N 33/50; G01N 33/543

(52) U.S. Cl. 702/19; 436/518

(57) ABSTRACT

A system and method capture the three-dimensional shape and functionality of molecules by the analysis of relevant intramolecular distances to generate a short and descriptive pharmacophoric fingerprint for each molecule. These fingerprints can then be used for diversity analysis, clustering, or database searching.

### CROSS REFERENCE TO RELATED APPLICATION

This application claims priority from U.S. Provisional Serial No. 60/253,835, filed Nov. 29, 2000, and which is expressly incorporated herein by reference.

### BACKGROUND OF THE INVENTION

Diversity is the measure of difference between elements in a set. This descriptive concept becomes quantitative when differences are numerically defined for a specific purpose. Whether qualitative or numerical, the concept of diversity (and its opposite, similarity) is significant to the ability to simplify matters through categorization.

In the field of drug discovery, it can be difficult to empirically validate models; consequently categorization is often the only available means of treating data scientifically. One such case is that of small molecules; given that it is not hard to estimate 10¹⁰⁰ possibilities for small molecules (Walters et. al., Drug Disc. Today, 1998, p. 160), the overall properties of small molecules are generally treated through categorization. Thus, the concept of molecular diversity is an important tool for understanding small molecules and their activities.

Classification of small molecules in drug discovery usually aims at improving hit-rate in high throughput screenings. Recognition of small molecules by macromolecules is largely mediated by shape and functional complementarity; consequently, diversity methods that numerically describe molecules based on some representation of three-dimensional (3D) shape and functionality are of particular value.

Molecular diversity applications must handle a vast number of molecules, and therefore a two-dimensional (2D) binary fingerprint approach is often used in comparisons of large databases. 2D methods, however, suffer from several major disadvantages. Lack of information on the actual shape and the location of the functional groups, poor recognition of isomers, and insensitivity to conformational issues can all render topological fingerprints essentially useless for library design. Furthermore, combinatorial libraries are often composed of close scaffold analogs reacted with a series of building blocks along various projection vectors to scan receptor relevant diversity space. The products generated by such combinatorial syntheses can be representatives of unique 3D pharmacophores that are difficult if not impossible to differentiate by traditional 2D fingerprints.

An approach for dequantized surface complementarity diversity has recently been reported (Wintner et. al., J. Med. Chem., 2000, p. 1993). This approach compares molecules based on their ability to satisfy complementarity to protein surfaces. The model used by this approach enumerates all theoretical combinations of quantized small molecule and protein surfaces at a low resolution.

To date, numerous ways of quantifying the diversity of molecules have been developed. Most of these ways are based on the principle of using molecular properties such as functionality and connectivity as a basis for categorization (e.g., Potter and Matter, J. Med. Chem., 1998, p. 478).

One approach is based on simple atomic connectivity and detection of the presence or absence of relevant functional groups (in case of, for instance, 2D fingerprints). This method, however, does not satisfactorily account for the 3D shapes of molecules and the specific location of the functional groups within the structure, which are some of the most critical aspects of a molecule's ability to bind to a macromolecule (e.g., Patterson, Cramer, Ferguson, Clark and Weinberger, J. Med. Chem. 1996, 39, p. 3049.) This approach also does not include many low energy states (conformations) for the molecules which gives rise to inadequate sampling of potential binding modes.

Another approach computes a surface (for instance, a solvent accessible or van der Waals surface) of the molecules, and matches and ranks pairwise similarities based on the ability of one surface to mimic the other. The entire process, however, needs to be repeated for all pairwise similarity measures (see, e.g., Mount et al., J. Med. Chem., 1999, p. 60, or Jain, J. Comp-Aided Mol. Design, 2000, p. 199).

Yet another approach registers all combinations of 3-point or 4-point pharmacophore points to create a binary fingerprint file as a representation of molecular properties (similar to 2D fingerprints). Pharmacophores are molecular properties, such as hydrophobic, H-bond donor, H-bond acceptor, and negatively or positively charged and polarizable moieties, all of which are believed to be of great relevance in the binding event of a small molecule to a macromolecule. The number of pharmacophore points for typical drug molecules can be significantly higher than three or four, however, and the fingerprint bins are distance-range dependent, giving rise to errors when a small deviation in distances renders similar properties into separate bins (see, e.g., Mason, et al., J. Med. Chem., 1999, p. 3251).

The ability to bind to a small set of natural proteins can be used as a basis for categorization (see, e.g., Dixon and Villar, J. Chem. Inf Comput. Sci., 1998, p.1192). While these methods of diversity calculation, often called affinity fingerprinting, can successfully categorize 3D molecular shape, they are limited to areas of diversity for which binding proteins have been isolated.

### SUMMARY OF THE INVENTION

The system and method of the present invention effectively captures the 3D shape and functionality of molecules by the analysis of relevant intramolecular distances to generate a short and descriptive pharmacophoric fingerprint for each molecule. These fingerprints can then be used for diversity analysis, clustering, or database searching. Conformational sampling is carried out when needed by the means of molecular dynamics.

The method of the present invention uses pairwise distances between a defined set of atoms based on shape and pharmacophore type to characterize a molecule. Shape is captured by pairwise distances between all heavy atoms of the molecule. All other properties, such as hydrophobes, H-bond donors, H-bond acceptors, negatively charged, and positively charged, are described by distances between the atoms that possess the particular property and all heavy atoms of the molecule. In this fashion, a relative position of all pharmacophore features is mapped on the overall shape of the molecule. In other words, the method considers the location of the atoms within the molecule in relation to the overall shape of the molecule (which can be described by the positions of all heavy atoms). If the relative location of the same property for two different molecules is similar but the overall shapes are different, the method yields a low similarity value.

Distance values between two atoms can be attained based on a single conformation of the molecule or as an average of distances derived from several conformations of the molecule obtained by a conformational search method such as molecular dynamics. Investigation of distance plots for test molecules revealed that very short distances add only noise to the data because bond distances and three-atom angles are by nature highly redundant within organic compounds. All distances below a threshold, such as 3 angstroms, are removed before analysis. Because the method works in distance space, the frame of reference for every molecule is internal and, therefore, no pairwise alignment is necessary when molecules are compared. The set of distances that represent a particular property are sorted by magnitude to yield a distance related plot for each molecule.

When numerically characterized, the atomic distance plots thus generated can express molecular recognition features. For each molecule, characterization values are extracted from the distance plots of each distance/property type to yield a final string termed here a total pharmacophore diversity (TPD) fingerprint. Characterization values may include slopes, intercepts, parameters of linear and nonlinear functions fitted on the distance plots, distance values, and number of distance values. The TPD fingerprints can then be viewed as coordinates in a multidimensional space, where the number of dimension equals the number of fingerprint values in the string.

Dissimilarity between molecules can be related to their weighted distance in this space: the farther apart the molecules are, the more dissimilar they are. Different pharmacophore types may be weighted according to user-defined criteria depending on the application and depending on the user's experience as to what weights are appropriate. Weightings can be applied to the parameters that characterize the fingerprint, such as providing a high weighting for the slope and a lower weighting for an intercept, or vice versa, and weightings can be used for the shape curves and the curves for properties.

The diversity method of the present invention overcomes shortfalls of various known similarity methods and preferably includes one or more of the following benefits:

(1) it generates a short shape and property related fingerprint file for every molecule;

(2) the flexible format allows for the addition of new properties if needed;

(3) the description of every property is continuous, and therefore no errors can arise from digital binning process;

(4) a fingerprint file describes the properties of a molecule not relative to comparison with any other molecule, and therefore calculation thereof needs to be carried out only once for every molecule;

(5) it considers an ensemble of all heavy atoms to encompass the total number of pharmacophores (by projecting the location of the property to the surface as described earlier) as opposed to a few pharmacophore points considered by other methods;

(6) molecules are compared without the need for alignment because the method works in distance space;

(7) fingerprint files can be created with or without conformational search (a particularly useful application of this type is when a binding conformation of a known ligand derived from, for instance, a crystal structure is evaluated with no conformational search to give a fingerprint that can be compared to that of a series of molecules evaluated with conformational search; the most similar structures identified by this method will not only have similar pharmacophore features but also preferred conformations close to the binding conformation of the known ligand);

(8) similarity values are first obtained for all included functionality separately and then combined per user instructions; if a binding feature is suspected to be of particular relevance in a given study, its contribution to the overall similarity can be weighted accordingly or can be looked at separately; and

(9) as a distance based method, the system incorporates information on both the overall molecular shape (long distances, above 6 angstroms) and the significant topological differences (shorter distances, below 6 angstroms) at the same time.

Other features and advantages will become apparent from the following detailed description, drawings, and claims.

### BRIEF DESCRIPTION OF THE DRAWINGS

FIG. 1 shows representations of two molecules that are compared to determine the similarity and diversity.

FIGS. 2A and 2B shows an example for obtaining a distance map for an H-bond acceptor oxygen for a structure.

FIG. 3 is a screen shot showing a fingerprint file according to the present invention.

FIG. 4 has graphs showing distance curves in decreasing order for four molecules that represent two different classes of ligands with the first graph showing a representation of shape and a second graph for H-bond acceptors.

FIG. 5 shows graphs of molecules and the chemical structures of those molecules.

FIG. 6 is a graph of a similarity histogram showing values obtained using the system and method of the present invention.

FIG. 7 is a graph of a cumulative histogram arranged by similarity values obtained by the system of the present invention.

### DETAILED DESCRIPTION

The concept of pharmacophore recognition during the binding event of a small molecule to a macromolecule, such as a protein, has been appreciated for many years in the scientific literature. An ever-increasing number of protein-ligand co-crystal structures has further helped the understanding of molecular recognition. The presence of important pharmacophore points in a correct arrangement is often required for a small molecule to bind to its target in order to satisfy functional compatibility. In addition to pharmacophore points, matching surface-to-surface contact between ligand and target established along the surface of the small molecule is critical for tight binding; that is, full shape and functional compatibility is necessary. Incompatibility in shape and/or function where close contacts exist may lead to significant loss of binding affinity even if the traditional pharmacophore point requirements are satisfied. This means that an entire molecule should be considered by the diversity method as a whole. The contribution of different parts of the small ligand to the free energy of binding varies, but incompatibility must be penalized to get meaningful predictions.

For any molecular property type, such as hydrophobic, H-bond donor, H-bond acceptor, and negatively or positively charged and polarizable moieties, the TPD system of the present invention calculates distances between every atom that possesses the property to all other heavy (non-hydrogen) atoms of the molecule. The system thus considers the location of the atoms within the molecule in relation to its position and the overall shape of the molecule, which can be described by the positions of all heavy atoms. If the relative location of the same property for two different molecules is similar but the overall shapes are different, the system yields a low similarity value.

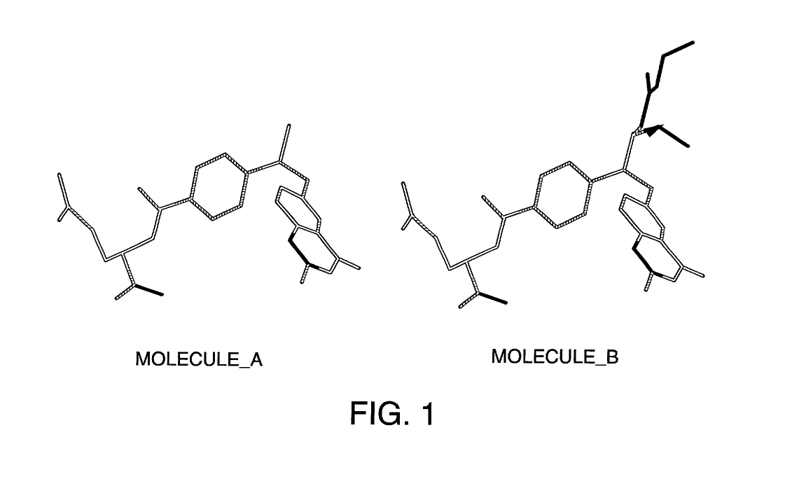

FIG. 1 shows two molecules for comparison. If only a few (such as 3 or 4) pharmacophore points are considered, the two molecules may look similar even though they cannot bind to the same binding site due to shape incompatibility for one molecule which does not exist in the other molecule.

As shown in FIG. 1, Molecule A has three binding features (negatively charged; hydrophobic; and positively charged). Molecule B has the same three features in the same relative orientation as seen in Molecule A, but Molecule B also contains a surface that is not present in Molecule A. That extra surface can prevent Molecule B from binding to a tight surface that Molecule A just fits into (as tight binders do). If only the three pharmacophore points were considered, the two molecules could look very similar (or even identical), but the method of the present invention, by further considering the overall shape, yields a relatively low similarity value.

In an embodiment of the present invention, pairwise distances are calculated between defined sets of atoms. The defined set of atoms varies with pharmacophore types, but is obtained using the same principles. Shape is captured by an ensemble of pairwise distances between all heavy atoms of the molecule. All other properties are captured by an ensemble of distances between the atom(s) that possesses the particular property and all heavy atoms of the molecule.

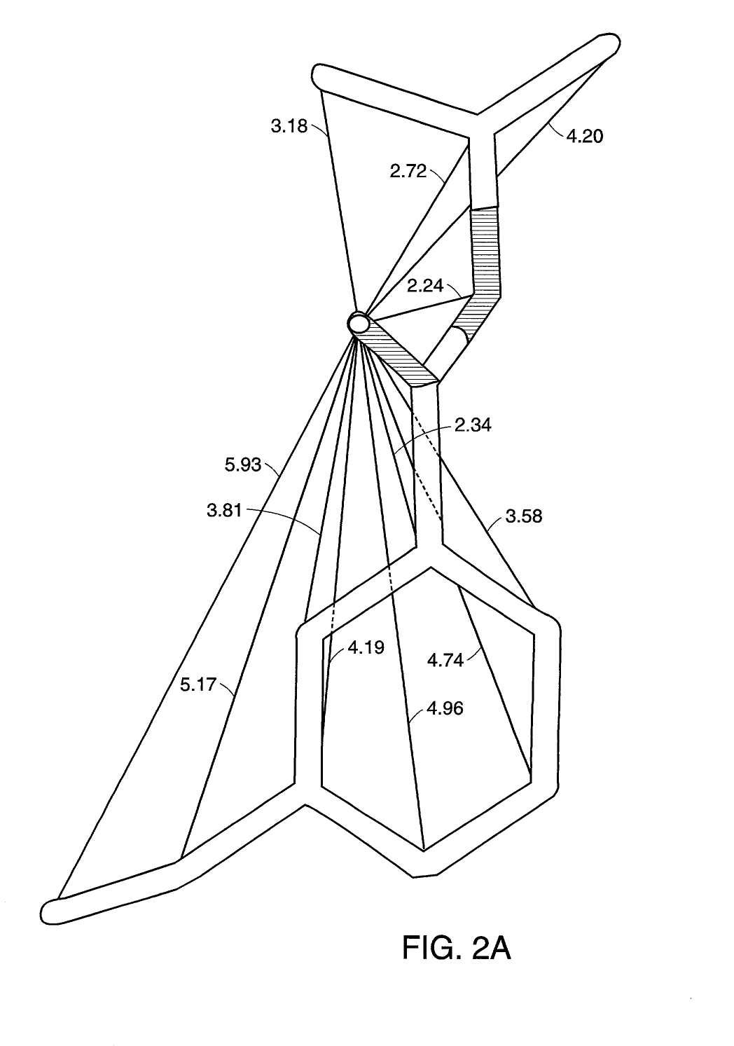

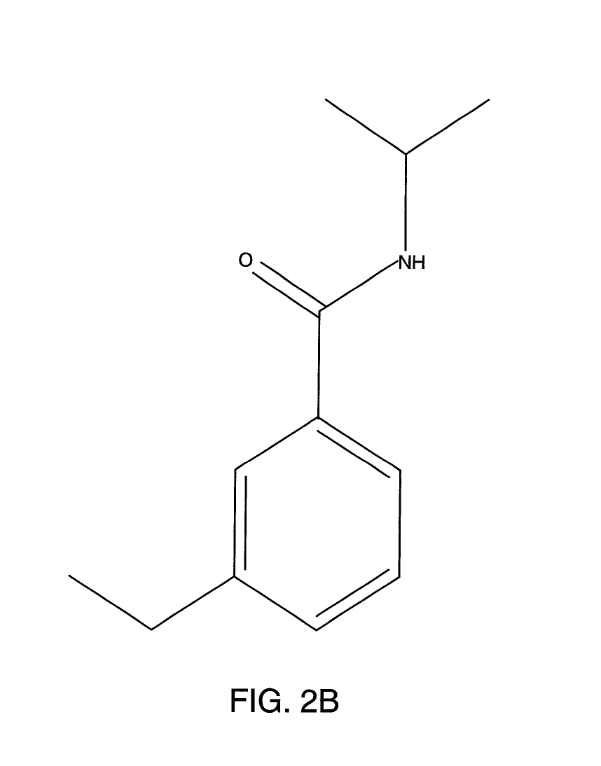

FIG. 2A shows an example of a distance map for an H-bond acceptor oxygen for the structure shown in FIG. 2B. That is, the map is of the distances from the H-bond acceptor oxygen to the other heavy atoms as shown. By doing so, the relative position(s) of the property is mapped on the overall shape of the molecule. Distance values between two atoms can be attained based on a single conformation of the molecule, or as an average of distances present in several conformations of the molecule obtained by a conformational search method, preferably molecular dynamics. Because the system works in distance space, the frame of reference for every molecule is internal and, therefore, no pairwise alignment is necessary when molecules are compared.

The set of distances that represent a particular property are processed by a method (described below) to yield descriptive fingerprint values. Distance values between all heavy atoms are computed and stored. As shown in FIG. 2A for one pharmacophore type, individual sets of distances between a pharmacophore type and all heavy atoms are obtained by knowledge-based methods separately for every pharmacophore type. The rules that render a particular heavy atom into a pharmacophore class are based on interactions commonly observed in molecular complexes and are well understood in terms of energetic contribution. New rules can easily be added to the system and method of the present invention.

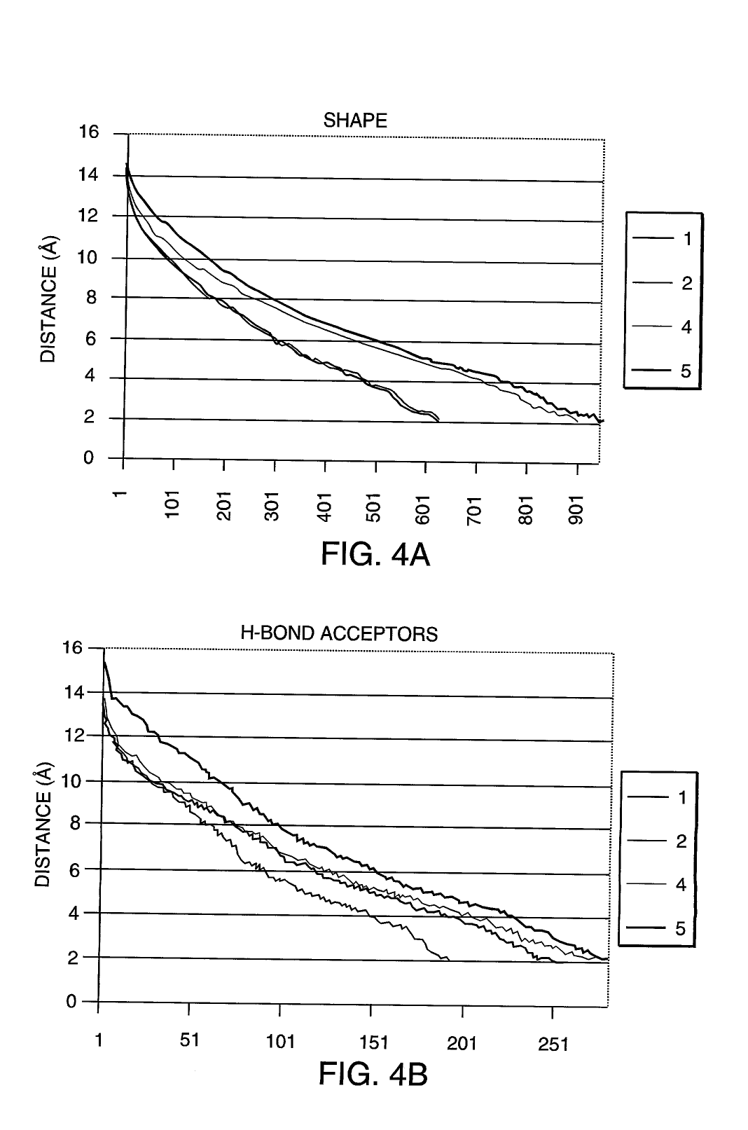

For the set of all heavy atoms and for every given pharmacophore type, distances are sorted in increasing or decreasing order to yield a curve as shown, for example, in FIG. 4. Thus, every molecule has a curve for the distances between its heavy atoms, to characterize the shape, and a curve for the distances between its heavy atoms and each pharmacophore type such as H-bond acceptors as shown in FIG. 4.

The first graph in FIG. 4 shows curves representing the shape for four molecules. As shown, two of the molecules have about 900 pairs of distances, and the other two molecules have about 600 pairs of distances. The distances are arranged to have a declining curve. As shown in FIG. 4, the distances have a minimum of 2 angstroms to the part of the curve. The curve representing the shape is generated from the pairwise relationship of all atoms in the molecule. If there are n atoms used for distance measurements, the number of possible pairwise distances is (n)(n−1)/2. The actual number will typically be less because of the minimum threshold for distances, e.g., a 2 Angstrom or 3 Angstrom minimum, below which the distances are ignored. The distances could be arranged with an ascending curve, or the x- and y-axes could be reversed.

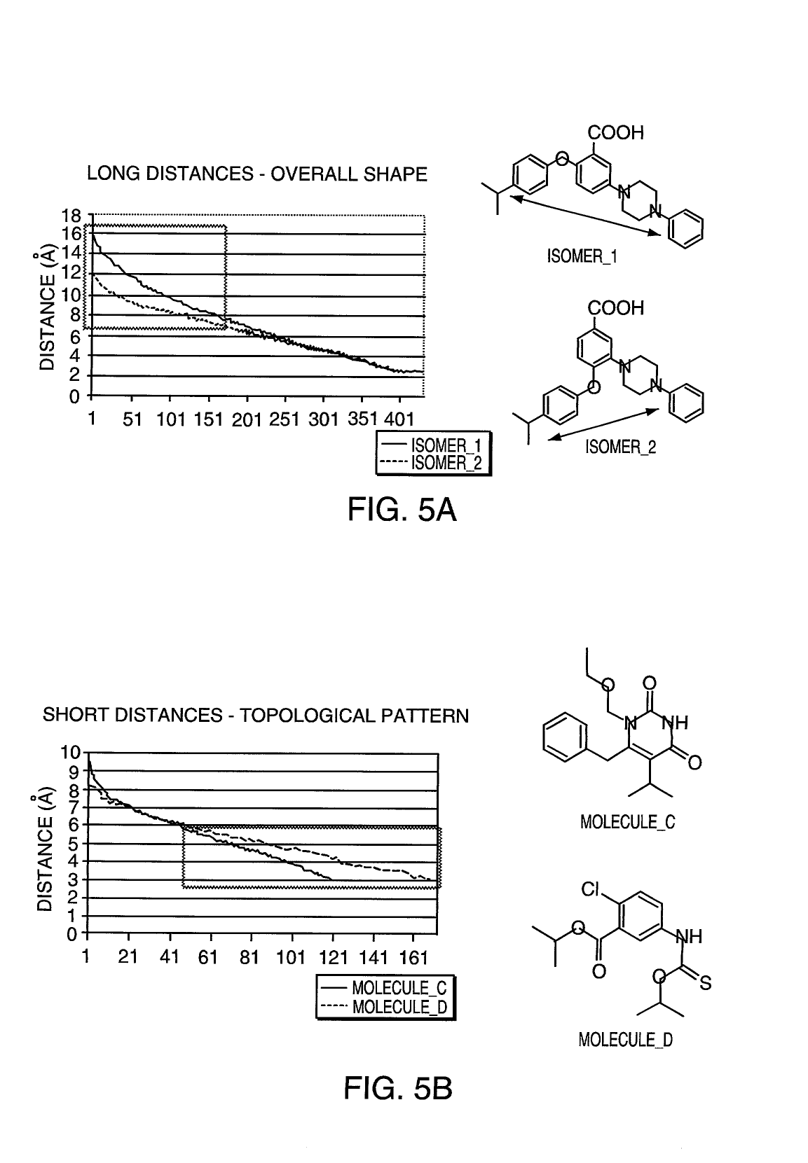

For the properties, such as H-bond acceptors, as shown in the second graph in FIG. 4, there will be fewer distances, namely a maximum of (m)(n−1) if there are n atoms in total and m atoms that possess H-bond donor properties. FIG. 5 also shows graphs and corresponding chemical drawings for certain molecules.

Each individual distance curve of the type shown in FIG. 4 or FIG. 5 can then be characterized by parameters or values that mathematically describe the curve, such as first or higher order derivatives (slope) or intercepts. To give a highly simplified example, if the curves were made to be the best linear fit (y=mx+b), they could be characterized by a slope and a y-intercept. For more complex curves, additional numbers will be used.

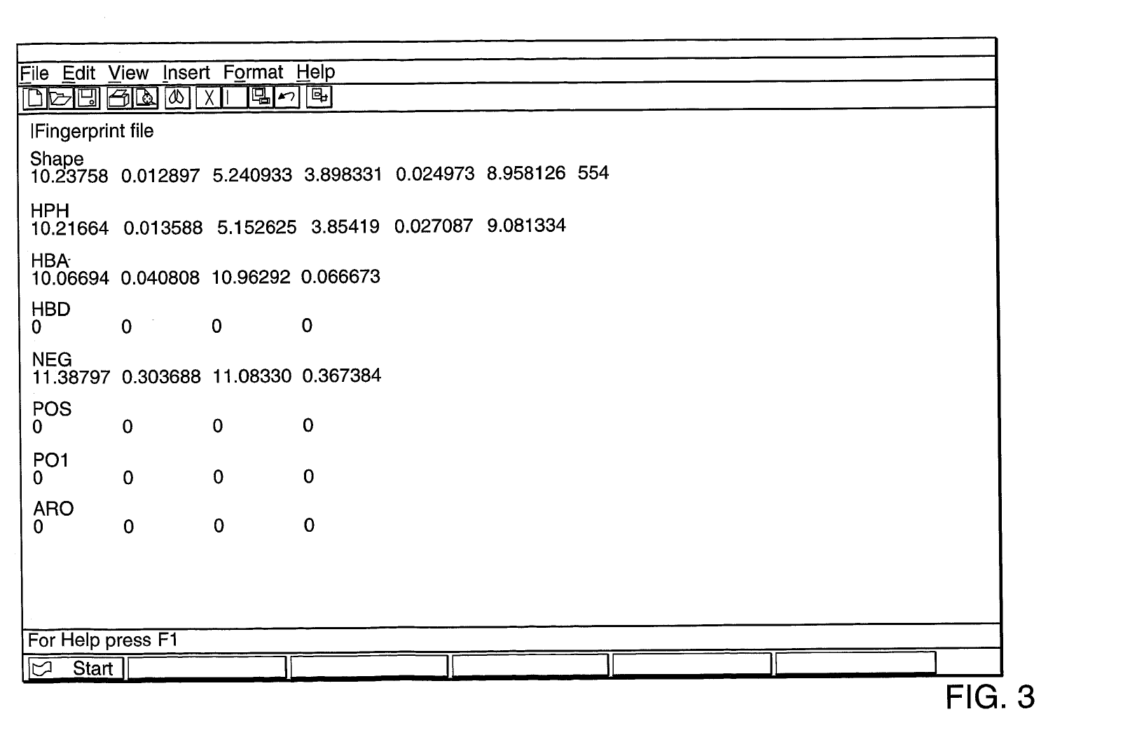

The system fingerprint of a molecule is thus a set of the list of values that characterize each curve. The fingerprint values describe a particular property and are stored in a fingerprint file, which is a binary or text file that contains numbers that describe every property considered by the system of the present invention, such as the file shown in FIG. 3. This file shows numerical representations for the shape, hydrophobes, H-bond acceptor, and negatively charged, and it also reveals that no H-bond acceptor, positively charged, polarizable and aromatic features are present in the molecule.

These fingerprints are thus represented by continuous graphs, unlike conventional binary fingerprints used by 2D approaches and 3-point and 4-point pharmacophore methods. Thus, the fingerprint values are numbers that can have values other than one or zero, while traditional methods generally produce ones and zeros only. The use of continuous fingerprints has certain advantages. First, in a binary fingerprint method, once a fingerprint value is set to 1 (meaning that the feature described by the given bin is present), it remains 1 even if there is more than one occurrence of that feature. According to this embodiment of the present invention, multiple occurrences of similar or identical features results in a shift of the property function curves and very different fingerprint values because the fingerprints are designed to characterize the curves.

A second advantage of using continuous fingerprints is that the binning process used by binary fingerprints is digital, meaning that the feature described by a given bin has to fit into bin limits, or else it will set another bin to one. This gives rise to errors not present in the continuous fingerprints.

To illustrate the effect of digital error, let us assume that bin 1 accounts for a distance between 3.0 and 3.8 angstroms for a pair of two H-bond donor atoms and bin 2 accounts for a distance between 3.8 and 4.6 angstroms for a pair of two H-bond donor atoms. If two similar molecules contain two H-bond donors with distances between them of 3.75 for molecule 1 and 3.82 for molecule 2, respectively, molecule 1 will set bin 1 to 1 and leave bin 2 as zero while molecule 2 will set bin 2 to 1 and leave bin 1 as zero in a digital fingerprinting method. For the H-bond donor feature that would result in much underestimated similarity between molecule 1 and molecule 2 by a digital binary method, while the system of the present invention has values that contain no error derived from such a binning processes.

The fingerprint values can be viewed as coordinates in a multidimensional space, where the number of dimension equals the number of fingerprint values. For details on using multidimensional space, see, for example, Pearlman and Smith, J. Chem. Inf. Comput. Sci. 1999, 39, p. 28.

Thus, a dissimilarity between molecules can be related to their distance in the multidimensional space. The farther the molecules are in the property space, the more dissimilar they are. Dissimilarity (or similarity) values are obtained separately for each of the pharmacophore types. Finally, the simple or custom weighted averaging of the shape and property similarity values yields the overall similarity number that numerically defines the capability of two molecules to bind to the same surface presented by a macromolecule.

A first method generates fingerprint files. Distance values between all heavy atoms are computed and stored first. Individual sets of distances between a pharmacophore type and all heavy atoms are obtained by knowledge-based methods for every pharmacophore type separately. Rules that render a particular heavy atom into a pharmacophore class are based on interactions commonly observed in molecular complexes and are well understood in terms of energetic contribution. New rules can be added as they become available. For the all heavy atom set and for every given pharmacophore type, the distances are sorted in increasing or decreasing order to yield a curve. Thus, every molecule has a curve for the distances between its heavy atoms and a curve for the distances between its heavy atoms and each pharmacophore type. Each individual distance curve is then characterized by parameters or values that mathematically describe the curve, thereby yielding values. The resulting fingerprint of a molecule is the set of the list of values that characterize each curve.

The first method includes the following steps:

(1) Read in coordinates for all heavy atoms into matrix1 for each conformation separately from a file that describes a molecule. This information can come from one of a number of common file formats (such as MDL's SD or RD format, or Tripos's MOL or MOL2 format). A molecule file may contain one or more conformations of the same molecule in a single file.

(2) Find all atoms in matrix1 that are to be considered by the defined property rules for property no. 1 to give atom list list1_prop1.

(3) Find all atoms in matrix1 that pass filters to give atom list list2_prop1. Filters applied here may include, but are not limited to, removal of atoms connected to atoms in list1_prop1 by a chemical bond, or atoms that produce distances below certain length

(4) Calculate distances between each atom in atom list list1_prop1 and all atoms in list2_prop1 for each conformation separately. Average distances for every atom pair if more than one conformation is present to give a final list of distances for property no. 1.

(5) Sort all distances from step 4 in increasing (or decreasing) order of magnitude.

(6) Repeat the process starting with step 1 or step 2 for all properties to be considered. The properties may include, without limitation:

Acidic moieties

Basic moieties

Moieties of formal positive charge

Moieties of formal negative charge

Moieties of partial positive charge

Moieties of partial negative charge

Hydrophobic moieties

Polarizable moieties

Hydrogen-bond donor moieties

Hydrogen-bond acceptor moieties

Aromatic moieties

(7) Characterize the distance curves obtained in step 6 to obtain values that describe the distance curve. Such values may include but are not limited to:

Slopes of linear regions

Slopes of nonlinear regions

Intercepts of linear regions

Intercepts of nonlinear regions

Parameters of functions obtained by linear regression

Parameters of functions obtained by nolinear regression

Parameters of functions obtained by polynomial fit

Maximum distance value

Distance value at any point of the curve

Number of distances

(8) Save as a list the values obtained in Step 7.

(9) Repeat steps 7 and 8 for all properties to be considered

(10) Save a fingerprint file for the molecule consisting of the set of all lists from step 9.

A second method provides for the evaluation of similarity or dissimilarity between two molecules using the fingerprints generated by the first method described above. Different methods and approaches can be used to compare two or more fingerprints. In one embodiment, a weighting approach based on molecule size and the number of occurrences of properties is applied to yield final similarity values as a measure of molecular similarity.

The numbers in the fingerprint files can be compared to obtain a curve-by-curve value representing similarity from one shape curve to another shape curve, and from one H-bond acceptor curve to another H-bond acceptor curve, and then those numbers relating to the similarity of each curve can be weighted for an overall similarity. The overall number can be a simple average of the curve-by-curve values, or these numbers can be weighted so that one or more counts for a higher percentage of the overall similarity score.

This second method includes the following steps:

(1) Read fingerprint files of molecules to be considered.

(2) Calculate a distance (difference) between pairs of molecules in a multidimensional space (number of dimensions equals number of fingerprints) for property no. 1.

(3) Apply weighting functions (which may be property dependent) on dissimilarity or similarity values for property no. 1 to obtain final similarity or dissimilarity values for all or a subset of all pairs of molecules.

(4) Repeat process starting with step 1 or step 2 for all properties.

By defining molecules in terms of characterization of their intramolecular distances, the total pharmacophore diversity method of the present invention allows:

(1) the creation of short pharmacophore based fingerprints that are continuous and not binary;

(2) the creation of short pharmacophore based fingerprints for every pharmacophore type separately;

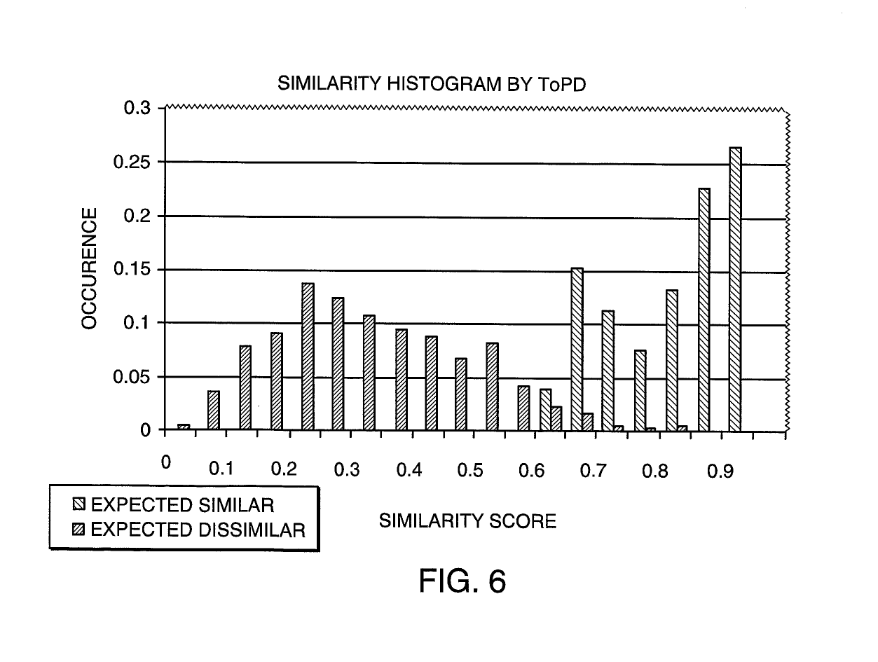

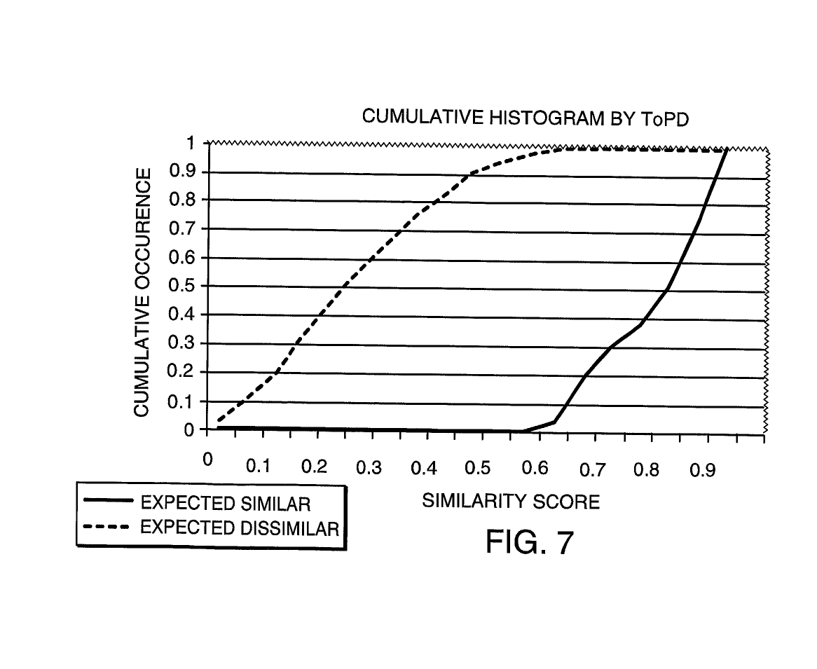

(3) the ability to compare the similarity or difference of molecules or sets of molecules based on their 3-dimensional shape and properties that are relevant for binding to macromolecules (FIGS. 6 and 7);

(4) assessment of molecular diversity of a set or sets of molecules based on their ability to interact with a macromolecule or another small molecule;

(5) clustering of compound files based on the fingerprints; and/or

(6) numerical prediction of binding ability of a molecule or sets of molecules as compared to a known small molecule ligand.

FIGS. 6 and 7 show the results of an experiment used to test the methods of the present invention. A number of molecules known to be similar and others believed to be dissimilar were compared. As shown particularly in FIG. 6, there are no expected similar molecules with a similarity score of 0.6 or less; and only a few expected dissimilar molecules with a similarity score above 0.6.

The method of the present invention may be implemented in software using a programmed general purpose computer or group of computers, or in a combination of hardware and software. The methods can also be carried out using application specific integrated circuitry (ASIC) or other specialty purpose processor. The computer would generally include some form of processor (general or specific purpose), volatile and non-volatile memory, and input/output functionality. Software or dedicated hardware would be responsive to input models for molecules for generating fingerprints, and responsive to multiple fingerprints for performing diversity analysis.

The fingerprints that are generated can be used to characterize a set of molecules, compare those molecules to each other, and used to determine likely binding affinity of a molecule to another molecule or a macromolecule. Thus the fingerprints can be stored in a database and used for comparison purposes and can also be used in a library to find molecules with desired characteristics.

The fingerprints generated according to this method were tested against a two dimensional fingerprinting approach known as “Unity Fingerprints”. The TPD fingerprints of the present invention performed similarly or better than the Unity Fingerprints over a number of different tests.

Having described the embodiments of the present invention, it should be apparent that modifications can be made without departing from the scope of the invention as defined by the appended claims.

What is claimed is:

1. A method of characterizing a molecule for determining molecular similarity or diversity including:
determining intramolecular distances between atoms of the molecule to characterize a shape of the molecule; and
for each of a group of properties, determining intramolecular distances between atoms with that property and other atoms of the molecule.

2. The method of claim 1, further comprising:
sorting distances by magnitude to create a curve;
numerically characterizing the curve; and
storing values representing the numerical characterization of the curve.

3. The method of claim 1, wherein the atoms that are used are heavy, non-hydrogen atoms.

4. The method of claim 1, wherein the atoms with a property are acidic atoms.

5. The method of claim 1, wherein the atoms with a property are basic atoms.

6. The method of claim 1, wherein the atoms with a property bear a formal positive charge.

7. The method of claim 1, wherein the atoms with a property bear a formal negative charge.

8. The method of claim 1, wherein the atoms with a property bear a partial positive charge.

9. The method of claim 1, wherein the atoms with a property bear a partial negative charge.

10. The method of claim 1, wherein the atoms with a property are hydrophobic atoms.

11. The method of claim 1, wherein the atoms with a property are polarizable atoms.

12. The method of claim 1, wherein the atoms with a property are hydrogen-bond donor atoms.

13. The method of claim 1, wherein the atoms with a property are hydrogen-bond acceptor atoms.

14. The method of claim 1, wherein the atoms with a property are aromatic atoms.

15. The method of claim 1, wherein the atoms with a property are all atoms.

16. The method of claim 1, wherein the method is performed for a number of molecules to create a series of fingerprints and the fingerprints are stored in a database.

17. The method of claim 16, wherein the fingerprints are compared on a pairwise basis to determine relative similarities to each other.

18. The method of claim 16, wherein in response to a determination that a molecule is desired with a particular shape and/or one or more properties, the method further comprising searching the fingerprints in the database to identify a molecule that has the desired shape and/or properties.

19. The method of claim 2, wherein the method is performed for a number of molecules to create a series of fingerprints and the fingerprints are stored in a database.

20. The method of claim 19, wherein the fingerprints are compared on a pairwise basis to determine relative similarities to each other.

21. The method of claim 19, wherein in response to a determination that a molecule is desired with a particular shape and/or one or more properties, the method further comprising searching the fingerprints in the database to identify a molecule that has the desired shape and/or properties.

22. The method of claim 16, further comprising searching the fingerprints to determine one or more molecules likely to bind to another molecule.

23. The method of claim 22, wherein said another molecule is a protein.

24. The method of claim 16, further comprising grouping together fingerprints considered sufficiently similar.

25. The method of claim 16, further comprising using a fingerprint to predict the binding ability of the molecule associated with that fingerprint to another molecule, as compared to the binding ability of another known molecule.

26. A method of characterizing a molecule for determining molecular similarity or diversity including:
determining intramolecular distances between atoms of the molecule to characterize a shape of the molecule;
sorting the distances to create a curve;
numerically characterizing the curve; and
storing values representing the numerical characterization of the curve and therefore of the molecule.

27. A method of characterizing a molecule for determining molecular similarity or diversity including:
for each of a group of properties, determining intramolecular distances between atoms with that property and other atoms of the molecule;
sorting the distances to create a curve;
numerically characterizing the curve; and
storing values representing the numerical characterization of the curve and therefore of the molecule.

28. The method of claim 27, wherein the property includes one or more of the following: acidic moieties, basic moieties, moieties of formal positive charge, moieties of formal negative charge, moieties of partial positive charge, moieties of partial negative charge, hydrophobic moieties, polarizable moieties, hydrogen-bond donor moieties, hydrogen-bond acceptor moieties, and aromatic moieties.
